(19) United States
(12) Patent Application Publication
(10) Pub. No.: US 2008/0243226 A1
(43) Pub. Date: Oct. 2, 2008

(54) IMPLANTABLE STENTS WITH RADIOPAQUE MARKERS AND METHODS FOR MANUFACTURING THE SAME

(76) Inventors: Jose E. Fernandez, Coral Springs, FL (US); Marc W. Litzenberg, Miami Shores, FL (US); Robert Lulo, Penbroke Pines, FL (US); Robert R. Slazas, Miami, FL (US); John H. Thinnes, Miami, FL (US)

Correspondence Address: COOK ALEX LTD, SUITE 2850, 200 WEST ADAMS STREET, CHICAGO, IL, 60606, US

(21) Appl. No.: 11/694,239

(22) Filed: Mar. 30, 2007

(51) Int. Cl. A61F 2/06

(52) U.S. Cl. 623/1.15

(57) ABSTRACT

Stents implantable within a body vessel include a strut with a receiving surface. The receiving surface has two openings passing through this surface. A radiopaque marker has a first prong received at least in part by one of the openings and a second prong received at least in part by the other opening. Approaches and methods are provided for enhancing engagement of the marker to the strut to prevent unintentional removal or dissociation of the marker from the strut.

### FIELD OF THE INVENTION

This invention relates to intravascular devices for implantation within a vessel of the body, and more particularly to a stent device which may be used in the treatment of blood vessel disorders. More particularly, this invention relates to stent devices having at least one radiopaque marker and methods for making the same.

### DESCRIPTION OF RELATED ART

Medical devices that can benefit from the present invention include those that are characterized by hollow interiors and that are introduced endoluminally and expand when deployed. These are devices that move or are moved between collapsed and expanded conditions or configurations for ease of deployment through catheters and introducers. Such devices are typically introduced to a diseased location within a body vessel (e.g., a stenosed section or an aneurysm) and may perform a variety of functions, including support and/or occlusion.

Endoluminal stents typically have a relatively open structure, with a plurality of interconnecting struts which define pores or openings in and/or through the surface that can allow for endothelialization and more permanent fixture of the stent within the vessel after implantation. Certain stents have an especially open structure in order to allow blood flow through the openings and to peripheral arteries after implantation of the stent adjacent to an aneurysm. Typically, the pores or openings are added by masking and/or etching techniques or laser- or water-jet cutting. Known stents include the Cordis Enterprise® line of self-expanding stents, which are described in numerous patents and published patent applications, including U.S. Pat. Nos. 6,612,012; 6,673,106; 6,818,013; 6,833,003; 6,955,685; 6,960,227; 7,001,422; and 7,037,331 and U.S. Patent Application Publication No. 2005/0234536, all of which are hereby incorporated by reference hereinto.

Stents have been developed with radiopaque markers to aid in the initial positioning of the stent within a body vessel and for visualization of the stent after deployment. Radiopaque markers facilitate the positioning of the stent within a blood vessel by allowing a physician to determine the exact location, size, and orientation of the stent under x-ray or fluoroscopy. These markers are typically formed of a radiopaque material such as tantalum, zirconium, titanium, or platinum U.S. Pat. No. 6,955,685 describes one known marking technique and is hereby incorporated by reference hereinto. This marking technique involves forming a stent strut with a portion having a plurality of threads. A coil of radiopaque material is wound around the threads and held in place by a UV adhesive.

One potential drawback of known marking techniques is their cost and complexity. For example, in techniques according to the foregoing description, the strut must be specially formed to include a threaded portion. Further, marker coils are very small, typically having a diameter on the order of 0.008 inch and a length on the order of one millimeter, so it may be difficult to manufacture and handle them or to properly wind them onto a threaded portion. These problems are further exacerbated by the fact that stents typically include a plurality of markers, so any manufacturing inefficiencies are multiplied.

Accordingly, a general aspect or object of the present invention is to provide an improved stent marking system that allows for increased manufacturing and assembly efficiency.

Other aspects, objects and advantages of the present invention, including the various features used in various combinations, will be understood from the following description according to preferred embodiments of the present invention, taken in conjunction with the drawings in which certain specific features are shown.

### SUMMARY

In accordance with an aspect of the present invention, a stent implantable within a body vessel comprises a strut with a receiving surface. A first opening passes through the receiving surface and a second opening is spaced from the first opening and also passes through the receiving surface. A radiopaque marker has a first prong received at least in part by the first opening and a second prong received at least in part by the second opening. Typically included is the use of one of various approaches to enhance engagement of the marker to the strut.

In accordance with another aspect of the present invention, a method is provided for securing a radiopaque marker to a stent implantable within a body vessel. The method involves providing a stent having a strut with a receiving surface. The receiving surface has a first opening passing therethrough and a second opening spaced from the first opening and also passing through the receiving surface. A radiopaque marker having a first prong with a free end and a second prong with a free end is also provided. At least a portion of the first prong is positioned in the first opening and at least a portion of the second prong is positioned in the second opening. Typically, an approach is followed for enhancing engagement of the marker to the strut.

### BRIEF DESCRIPTION OF THE DRAWINGS

FIG. 1 is a perspective view of a stent according to an aspect or embodiment of the present invention;

FIG. 2 is a detail view of a strut and radiopaque marker illustrated in FIG. 1;

FIG. 3 is a side elevational view of the strut and radiopaque marker of FIG. 2;

FIG. 4 is a perspective view of a pre-shaped radiopaque marker according to an aspect or embodiment of the present invention;

FIGS. 5A-5D are perspective views that illustrate an exemplary method of assembling an “unshaped” radiopaque marker and a strut;

FIG. 6 is a cross-sectional view of a strut and radiopaque marker according to another aspect or embodiment of the present invention;

FIG. 7 is a front perspective view of a strut according to yet another aspect or embodiment of the present invention;

FIG. 8 is a front perspective view of the strut of FIG. 7 receiving a radiopaque marker;

FIG. 9 is a rear perspective view of the strut and radiopaque marker of FIG. 8 and

FIG. 10 is a cross-sectional view of the strut and radiopaque marker of FIG. 7, taken through the line 10-10 of FIG. 8.

### DESCRIPTION OF ILLUSTRATED EMBODIMENTS

As required, detailed embodiments of the present invention are disclosed herein; however, it is to be understood that the disclosed embodiments are merely exemplary of the invention, which may be embodied in various forms. Therefore, specific details disclosed herein are not to be interpreted as limiting, but merely as a basis for the claims and as a representative basis for teaching one skilled in the art to variously employ the present invention in virtually any appropriate manner.

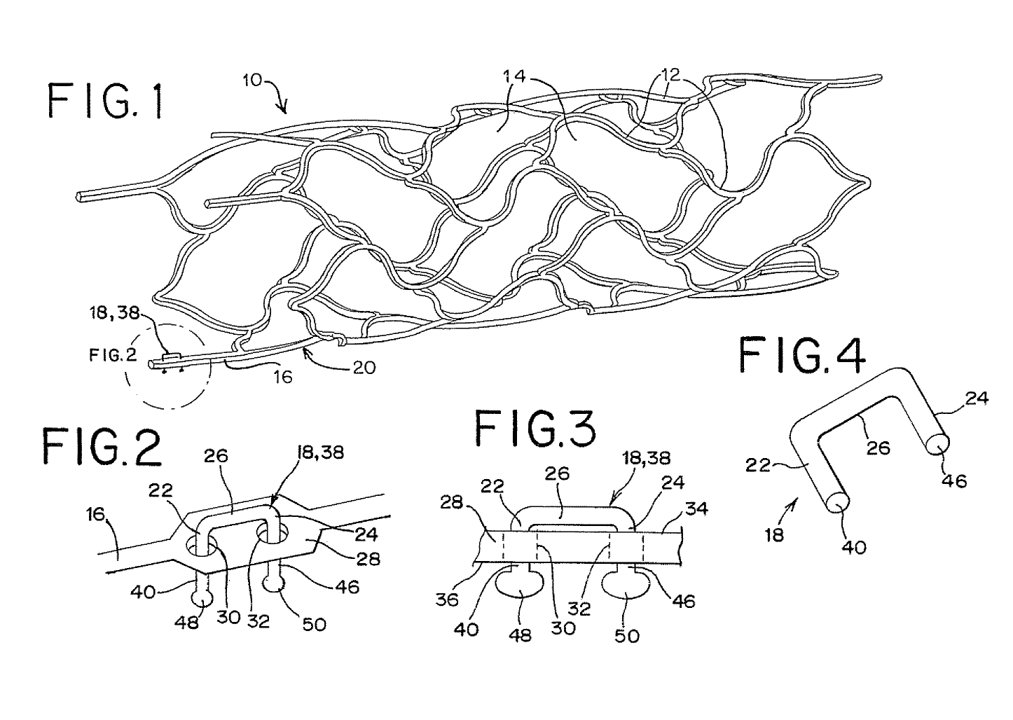

FIG. 1 shows a stent 10 which is implantable within a body vessel. The illustrated stent 10 is a substantially tubular device comprised of a plurality of interconnecting struts 12. The struts 12 may be formed by any of a number of methods including, but not limited to, laser cutting, water jet cutting, and etching. The struts 12 define a plurality of cells 14, which are deformable to allow the stent 10 to move between a small-diameter “compressed” or “delivery” condition (typically during manufacture and delivery) and a large-diameter “expanded” or “deployed” condition (during deployment and, in some cases, manufacture). A closed-cell configuration is shown in FIG. 1, but an open-cell configuration may also be employed without departing from the scope of the present invention. Stents according to the present invention may be self-expanding or balloon-expandable or partially self-expanding and partially balloon-expandable.

One strut 16 is illustrated with a radiopaque marker 18 according to the present invention. The strut 16 is shown at a proximal end 20 of the stent 10, but it will be appreciated that radiopaque markers according to the present invention may be incorporated into any strut. Further, a plurality of radiopaque markers may be used with a stent, either on different struts or on a single strut. In one embodiment, eight radiopaque markers may be used in combination with a stent, with four markers being associated with four separate struts at a proximal end of the stent and four markers being associated with four separate struts at a distal end of the stent. This may be preferred to more clearly define the front and rear portions of the stent for improved positioning with respect to a target site of a body vessel.

The strut 16 and marker 18 of FIG. 1 are shown in greater detail in FIGS. 2 and 3. FIG. 4 shows the pre-shaped marker 18 separate from the strut, in a pre-assembly condition. The marker 18 has a first elongated prong or leg 22 and a second elongated prong or leg 24. The illustrated prongs are substantially parallel and identical to each other, which may be preferred to simplify manufacture and assembly, but the prongs may have different configurations and be non-parallel without departing from the scope of the present invention.

The prongs are attached to each other by a crown 26. The illustrated crown 26 is a simple crossbeam connected to an end of each prong, with both prongs extending away therefrom in the same direction to give the marker 18 a generally U-shaped configuration. Such a configuration may be preferred to minimize the material requirements of the marker and simplify construction, but other crown shapes and marker configurations are within the scope of the present invention.

In the illustrated embodiments, the crown 26 extends beyond the surface of the strut 16, so the shape of the crown may be selected to provide a number of functions, such as acting as an engagement surface for a thin film mesh or screen member associated with the stent or a delivery or deployment device. For example, U.S. Pat. No. 6,955,685, previously incorporated by reference hereinto, describes radiopaque markers providing an engagement function during delivery of a stent to a body vessel, and the crown of radiopaque markers according to the present invention may be adapted to provide a similar function.

Regardless of the particular configuration of the marker, it is adapted for use in combination with a stent strut 16. A suitable strut 16 includes a receiving surface 28 having a first opening 30 and a second opening 32. The illustrated openings are generally aligned with each other along the length of the strut 16, but they may be arranged in other configurations without departing from the scope of the present invention. The receiving surface 28 may be enlarged with respect to the rest of the strut 16, as shown in FIG. 2, to provide a larger border around the openings. This may be preferred to minimize the risk of material failure at the openings, especially when the openings are arranged side-by-side, i.e., aligned with each other in a direction transverse to the length of the strut 16. Alternatively, the receiving surface 28 may have substantially the same dimensions as the rest of the strut 16, especially if the openings are relatively small or aligned along the length of the strut 16.

The first opening 30 and the second opening 32 pass through the receiving surface 28 from a front face 34 thereof to a rear face 36 thereof (FIG. 3). The openings may be formed by any method, though it may be advantageous to use the same method used to form the associated strut 16. For example, in one embodiment, the struts of the entire stent and the openings are formed by laser-cutting operations.

Each opening is configured to receive a prong of the marker 13, so the configuration and orientation of the openings typically are selected to match the configuration of the marker prongs, with the first opening 30 being keyed to the first prong 22 and the second opening 32 being keyed to the second prong 24. For example, in the embodiment of FIGS. 1-4, the prongs are substantially parallel to each other, so the openings are also substantially parallel to each other, comprising bores perpendicular or normal to the opposing faces of the receiving surface 28.

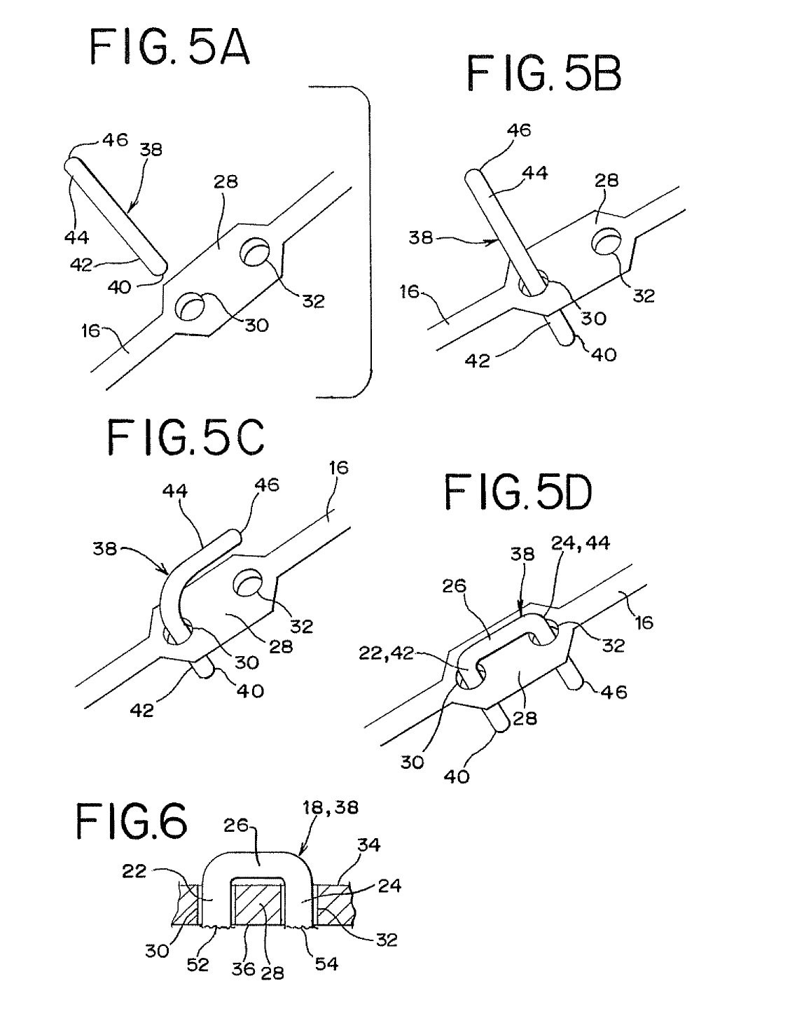

Alternatively, instead of providing openings adapted to match the prongs of a pre-shaped marker 18, the marker initially may be “unshaped” and adjustable to match the configuration of the openings. As used herein, the term “unshaped” refers to a marker with a shape that must be manipulated during the assembly process to define prongs receivable by the strut openings. One example of an “unshaped” marker is shown in FIG. 5A as a substantially straight wire and is generally designated at 38. A number of other “unshaped” configurations are also possible, and the embodiment of FIG. 5A is merely exemplary.

A number of methods of assembling an “unshaped” marker 38 onto a strut 16 are described herein with reference to FIGS. 5A-5D. In one method, the free end 40 of a first portion 42 of the marker 38 (the first portion 42 ultimately corresponding to the first prong 22) is passed through the first opening 30, in a downward motion with reference to the orientation of FIG. 5B. Thereafter, the marker 38 is bent to define an L-shape, as shown in FIG. 5C. The marker 38 is again bent from the L-shape to a U-shape (FIG. 5D) having a second portion 44 (ultimately corresponding to the second prong 24) and a free end 46 of the second portion 44 is passed through the second opening 32, in a downward motion with reference to the orientation of FIG. 5D.

According to another method of assembling an “unshaped” marker 38 onto a strut 16, the marker 38 is passed upwardly through the first opening 30, with reference to the orientation of FIG. 5B, leaving the free end 40 of the first portion 42 at the underside of the strut 16. Thereafter, the marker 38 is bent into an L-shape (FIG. 5C) and a U-shape (FIG. 5D) according to the foregoing description, and the free end 46 of the second portion 44 is passed downwardly through the second opening 32, with reference to the orientation of FIG. 5D. Any number of other assembly methods, which typically vary depending on the particular configuration of the “unshaped” marker and the strut, are also possible. Any of these methods may be employed without departing from the scope of the present invention. Furthermore, while the “unshaped” marker 38 of FIGS. 5A-5D is referred to herein as having first and second “portions” instead of “prongs,” it will be seen that the “portions” ultimately define “prongs” after proper manipulation and reshaping (FIG. 5D), so reference herein to markers having “prongs” or processing of a “prong” should be understood as encompassing both pre-shaped and “unshaped” markers.

Returning now to the strut 16, the diameter of each opening is greater than or equal to the diameter of the corresponding prong to allow the prongs to at least partially pass through the openings. When assembled, the free ends of the prongs extend beyond the rear face 36 of the receiving surface 28, while the crown 26 remains at the front face 34 thereof, as shown in FIG. 3.

Usually, an approach is taken to enhance engagement of the marker to the strut. For example, as noted herein, steps can be taken to secure the radiopaque marker 18, 38 to the strut 16. In one approach, if the diameter of each opening is substantially equal to the diameter of the corresponding prong, a press-fit or friction fit may be achieved to secure the marker 18, 38. If an opening diameter is greater than the diameter of the corresponding prong, other steps may be taken to enhance engagement of the marker and prevent inadvertent removal or dissociation of the marker 18, 38 from the strut 16.

In one exemplary method of enhancing engagement, the marker 18, 38 is secured to the strut 16. The free end 40 of the first prong 22 may include a bead 48 having a diameter greater than a diameter of the first opening 30, as shown in FIG. 3. In such an embodiment, the bead 48 prevents the free end 40 from passing through the first opening 30 from the rear face 36 to the front face 34, so the marker 18, 38 is essentially locked onto the strut 16. A single enlarged bead can be sufficient to secure the marker 18, 38 to the strut 16, but the free end 46 of the second prong 24 may also be provided with a bead 50 having a diameter greater than a diameter of the second opening 32 (FIG. 3) as an auxiliary fastening feature to enhance engagement of the marker to the strut. It may be considered advantageous to provide each free end with a bead, because the beads may be adapted to be more atraumatic than the free ends, which may be sharp as a result of the manufacturing process and/or due to their size and/or shape.

In one method of forming a bead, a prong free end is deformed to increase the diameter, thereby forming a bead. Suitable deformation methods will vary according to the material selected for the marker, but exemplary deformation methods include melting and swaging. In another method of providing a bead, a separate bead element may be affixed to a prong free end. The foregoing methods are merely exemplary and a bead may be formed by any method without departing from the scope of the present invention.

Regardless of the method of forming the bead, the bead may be formed before or after the associated free end is passed through the corresponding strut opening. It may be preferred to pass the associated free end through the corresponding opening before forming the bead, which avoids a step of passing the enlarged bead through the smaller opening. However, it may also be considered advantageous to form the bead prior to joining the marker and strut, as it may be more economical to fully form the marker in a single step, which avoids a separate step after the prongs have been positioned in the strut openings. If the bead is formed prior to assembly, it may be preferred to construct the receiving portion 28 of the strut from a material having shape memory properties, such as martensitic nitinol, to allow each opening to be enlarged during insertion of a bead therethrough and to thereafter return to a smaller diameter. In some instances, expansion of the receiving portion 28 by for example heating may achieve a similar effect for enhanced engagement of the marker to the stent.

In another exemplary method of securing the marker 18, 38 to the strut 16, one or both of the prongs may be adhered or otherwise bonded to a portion of the receiving surface 28 or the corresponding opening. Suitable bonding methods will vary depending on the material composition of the marker and the strut, and the selection of an appropriate bonding method is within the capabilities of one of ordinary skill in the art. By way of example, FIG. 5 shows a marker 18, 38 secured to a strut 16 by a seam welding operation. The first prong 22 is secured within the first opening 30 by a first weld 52 and the second prong 24 is secured within the second opening 32 by a second weld 54. A single weld is typically sufficient to secure the marker 18, 38 to the strut 16, but it may be preferred to seam weld each prong to eliminate the portion of the prongs extending beyond the rear face 36, thereby providing a relatively smooth surface.

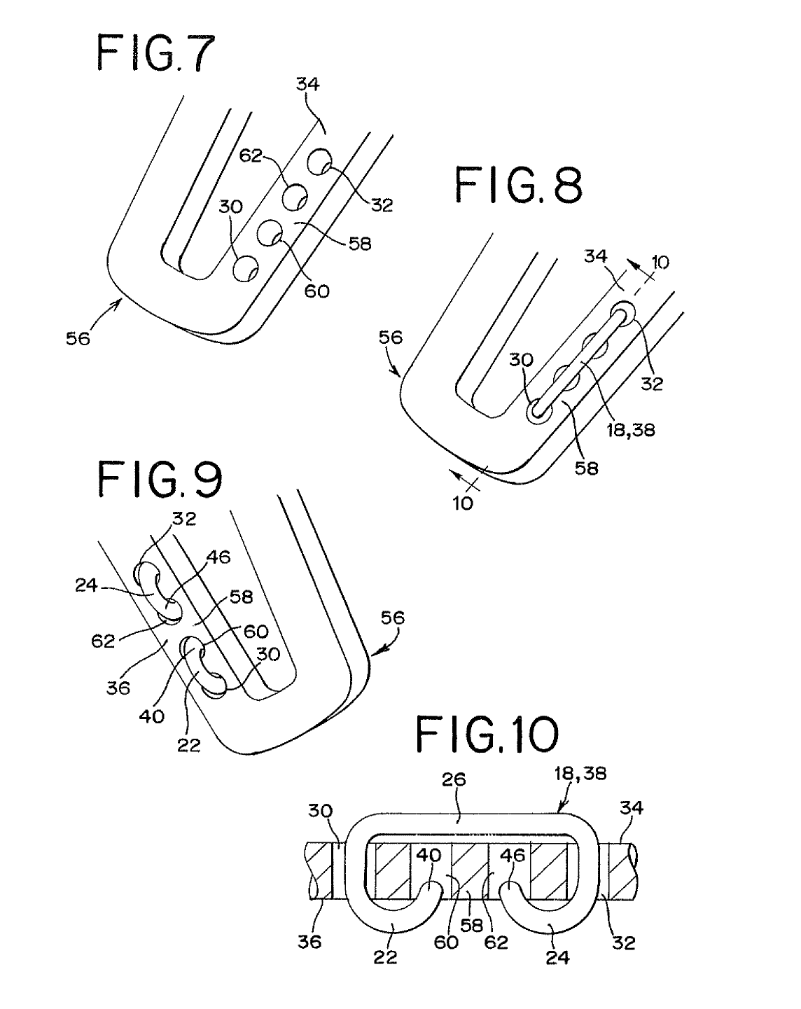

The receiving surface may be modified to accommodate yet another method of securing the marker to the strut. FIGS. 7-10 illustrate a strut 56 having an alternative receiving surface 58. In addition to a first opening 30 and a second opening 32, the receiving surface 58 includes a third opening 60 and a fourth opening 62. In the illustrated embodiment, the four openings are in a generally straight row, with the third opening 60 and the fourth opening 62 between the first opening 30 and the second opening 32, the third opening 60 adjacent to the first opening 30, and the fourth opening 62 adjacent to the second opening 32. Other configurations of the openings are within the scope of the present invention, but the illustrated embodiment may be preferred for use with a generally U-shaped radiopaque marker.

At least a portion of the first prong 22 passes through the first opening 30 and at least a portion of the second prong 24 passes through the second opening 32, as shown in FIGS. 8 and 10 and in accordance with the foregoing description of the embodiments of FIGS. 1-6. Thereafter, the first prong 22 is bent until the free end 40 thereof extends into the third opening 60 and the second prong 24 is bent until the free end 46 thereof extends into the fourth opening 62, as shown in FIGS. 9 and 10. As perhaps best shown in FIG. 10, the final configuration of the marker 18, 38 may be understood with reference to a clinched staple. Hence, bending the prongs serves to secure the marker 18, 38 to the strut 56, while providing the additional benefit of directing the free ends, which may be sharp due to the manufacturing process, away from the outside environment.

The prongs may be bent by any method, such as by gripping the prong with a tool and twisting it until the free end extends into the appropriate opening. In another exemplary method, a forming fixture may be provided with shaped cavities into which the free ends are pressed and thereby bent, similar to the operation of the anvil of a typical stapler.

As shown in FIG. 9, the free ends of the prongs need not extend fully into the third and fourth openings. Accordingly, the third and fourth openings need not extend completely through the receiving surface 58 and one or both may be provided as a bore in the rear face 36 that stops short of the front face 34.

In general, the material composition of struts/stents and radiopaque markers according to the present invention is in no way limited and may include any materials known in the art as suitable for stents and/or struts, as well as materials which may hereafter become known in the art. By way of example, the stent and struts may be comprised of biocompatible metals, such as stainless steel or a nitinol material, or biocompatible polymers. By way of further example, a suitable material for the marker is detectable under x-ray, fluoroscopy and the like, and includes, but is not limited to, platinum, gold, tantalum, zirconium and other materials having radiopaque properties. If the stent is constructed of a biodegradable polymeric material, it may be preferred to also provide the marker as a biodegradable material to avoid degradation of the stent and the possibility, no matter how unlikely, of subsequent migration of a free marker through the vasculature.

Those of ordinary skill in the art will appreciate that certain embodiments of the present invention may further restrict the scope of suitable materials. For example, the embodiment of FIG. 6 involves welding the marker to the stent, so care should be taken to select materials that are weldable to each other. Similarly, the embodiment of FIGS. 7-10 involves bending the prongs of the marker, so the marker material should be suitable for such manipulation.

A stent according to the present invention may be used according to any of a number of methods well-known to those of ordinary skill in the art. In one exemplary manner of use, the stent is inserted into the distal end of an introducer (not shown). The stent may be mounted about a guidewire or a balloon catheter before being inserted into the introducer

When the stent has been properly loaded according to an introducer approach, the introducer is moved into the interior of a body vessel and positioned adjacent to a region of the vessel which is to be occluded. Thereafter, the stent is ejected from the introducer and into the target region. If the stent is not self-expanding, then a balloon is expanded to force the stent against the wall of the vessel. The markers assist in properly positioning the stent during deployment and in locating the stent after deployment.

It will be understood that the embodiments of the present invention which have been described are illustrative of some of the applications of the principles of the present invention. Numerous modifications may be made by those skilled in the art without departing from the true spirit and scope of the invention, including those combinations of features that are individually disclosed or claimed herein.

1. A stent implantable within a body vessel, comprising:
a strut with a receiving surface, a first opening passing through the receiving surface, and a second opening spaced from the first opening and passing through the receiving surface; and
a radiopaque marker having a first prong received at least in part by the first opening and a second prong received at least in part by the second opening.

2. The stent of claim 1, wherein the radiopaque marker is generally U-shaped.

3. The stent of claim 1, wherein the first prong includes a free end having a bead with a diameter greater than a diameter of the first opening to prevent removal of the radiopaque marker from the strut.

4. The stent of claim 3, wherein the second prong includes a free end having a bead with a diameter greater than a diameter of the second opening to prevent removal of the radiopaque marker from the strut.

5. The stent of claim 1, further comprising a first weld associated with the first prong to secure the first prong within the first opening.

6. The stent of claim 5, further comprising a second weld associated with the second prong to secure the second prong within the second opening.

7. The stent of claim 1, further comprising a third opening passing at least partially through the receiving surface, wherein the first prong includes a free end received by the third opening to prevent removal of the radiopaque marker from the strut.

8. The stent of claim 7, further comprising a fourth opening passing at least partially through the receiving surface, wherein the second prong includes a free end received by the fourth opening to prevent removal of the radiopaque marker from the strut.

9. The stent of claim 8, wherein third opening and the fourth opening are positioned between the first opening and the second opening, with the third opening being adjacent to the first opening, and the fourth opening being adjacent to the second opening.

10. The stent of claim 9, wherein said openings are configured in a generally straight row.

11. The stent of claim 1, wherein at least a part of at least one of said first prong and second prong is configured to enhance engagement between same and said opening of the strut associated therewith in order to maintain assembly of the marker to the strut.

12. A method of securing a radiopaque marker to a stent implantable within a body vessel, comprising:
providing a stent having a strut with a receiving surface, a first opening passing through the receiving surface, and a second opening spaced from the first opening and passing through the receiving surface;
providing a radiopaque marker having a first prong with a free end and a second prong with a free end;
positioning at least a portion of the first prong in the first opening; and
positioning at least a portion of the second prong in the second opening.

13. The method of claim 12, wherein said providing a radiopaque marker includes providing a generally U-shaped radiopaque marker.

14. The method of claim 12, further comprising bending the radiopaque marker to define said second prong.

15. The method of claim 14, wherein said bending the radiopaque marker to define said second prong follows said positioning at least a portion of the first prong in the first opening.

16. The method of claim 12, further comprising increasing the diameter of at least one of said free ends to prevent removal of the radiopaque marker from the strut.

17. The method of claim 12, further comprising increasing a diameter of each of said free ends to prevent removal of the radiopaque marker from the strut.

18. The method of claim 12, further comprising welding said first prong to one of said first opening and said receiving surface to prevent removal of the radiopaque marker from the strut.

19. The method of claim 12, further comprising welding said first prong to one of said first opening and said receiving surface and welding said second prong to one of said second opening and said receiving surface to prevent removal of the radiopaque marker from the strut.

20. The method of claim 12, wherein said providing a stent includes providing a third opening passing at least partially through the receiving surface, and further comprising positioning the free end of the first prong in the third opening to prevent removal of the radiopaque marker from the strut.

21. The method of claim 12, wherein said providing a stent includes providing a third opening passing at least partially through the receiving surface and a fourth opening passing at least partially through the receiving surface, and further comprising positioning the free end of the first prong in the third opening and a step of positioning the free end of the second prong in the fourth opening to prevent removal of the radiopaque marker from the strut.
